(19) United States
(12) Patent Application Publication
(10) Pub. No.: US 2021/0401325 A1
(43) Pub. Date: Dec. 30, 2021

(54) GAIT MEASUREMENT SYSTEM, GAIT MEASUREMENT METHOD, AND PROGRAM STORAGE MEDIUM

(71) Applicant: NEC Corporation, Minato-ku, Tokyo (JP)

(72) Inventors: Kenichiro FUKUSHI, Tokyo (JP); Kentaro NAKAHARA, Tokyo (JP); Hiroshi KAJITANI, Tokyo (JP); Chenhui HUANG, Tokyo (JP)

(73) Assignee: NEC Corporation, Minato-ku, Tokyo (JP)

(21) Appl. No.: 17/294,490

(22) PCT Filed: Nov. 20, 2018
(86) PCT No.: PCT/JP2018/042807

§ 371 Date: May 17, 2021

(51) Int. Cl. A61B 5/11; A61B 5/00

(52) U.S. Cl. CPC A61B 5/112; A61B 2562/0219; A61B 5/742

(57) ABSTRACT

A gait measurement system includes: a locus calculation device that detects at least one gait phase from acceleration data measured by an inertial measurement unit, calculates velocity data by subjecting the acceleration data to time integration, calculates correction amounts associated with each of the gait phases using the gait phases and the velocity data, calculates correction velocity data by subtracting the correction amounts from the velocity data associated with each of the gait phases, and calculates locus data by subjecting the calculated correction velocity data to time integration; and an index calculation device that calculates a gait index, using the locus data calculated by the locus calculation device.

### TECHNICAL FIELD

The present invention relates to a gait measurement system, a gait measurement method, and a program for measuring gait.

### BACKGROUND ART

PTL 1 discloses a motion analysis device that estimates a walking state and a traveling state, using an acceleration sensor attached to a human body. The device disclosed in PTL 1 calculates a moving velocity or a moving distance, based on acceleration detected by an acceleration sensor attached to a human body. Generally, a value measured by an acceleration sensor has an error, and a measured value is not zero even in a case where the acceleration is zero. Therefore, an integrated error is included in a velocity calculation result being the value of integral of acceleration, and in a distance calculation result being the value of integral of velocity. This error, which is called “drift”, tends to increase with time. The device disclosed in PTL 1 prevent a decrease in accuracy due to the drift by assuming that the drift increases at a constant rate and integrating corrected acceleration obtained by subtracting from the acceleration a correction value designed to make the velocity at the end of measurement become zero.

PTL 2 discloses a walking distance meter that measures a walking distance, using an acceleration sensor attached to one ankle. The walking distance meter disclosed in PTL 2 sets an acceleration dead zone in which a portion having a digital conversion value of acceleration smaller than a certain value when the foot is in contact with the ground is recognized as “acceleration 0”. The walking distance meter disclosed in PTL 2 eliminates an error due to an offset of velocity data of the acceleration sensor, by resetting the value of integral of the digital conversion value of acceleration and a clock measurement time to 0 during the period in the acceleration dead zone. In this manner, the walking distance meter disclosed in PTL 2 eliminates the occurrence of an error due to a stride length.

### CITATION LIST

### Patent Literatures

[PTL 1] JP 2016-150193 A

[PTL 2] JPH 10-332418 A

### SUMMARY OF INVENTION

### Technical Problem

The device disclosed in PTL 1 can remove the drift that increases at a constant rate from the start till the end of measurement. However, the waveforms of the moving velocity and the moving distance actually calculated based on the values measured by the acceleration sensor show different characteristics between a stance phase and a swing phase, and the drift does not increase at a constant rate. Therefore, the device disclosed in PTL 1 cannot accurately detect changes in the drift, and has a problem of being incapable of sufficiently preventing a decrease in accuracy.

The walking distance meter disclosed in PTL 2 prevents the occurrence of drift due to integral during the foot contact period, by regarding the acceleration during the foot contact period as zero through threshold processing. However, the walking distance meter disclosed in PTL 2 does not take into account the drift that occurs during the swing phase in which the foot is not in contact with the ground, and therefore, has a problem of being incapable of sufficiently preventing a decrease in accuracy.

The present invention aims to solve the above problems, and provide a gait measurement system capable of measuring gait with high accuracy.

### Solution to Problem

A gait measurement system of one aspect of the present invention includes: a locus calculation device that detects at least one gait phase from acceleration data measured by an inertial measurement unit, calculates velocity data by subjecting the acceleration data to time integration, calculates correction amounts associated with each of the gait phases using the gait phases and the velocity data, calculates correction velocity data by subtracting the correction amounts from the velocity data associated with each of the gait phases, and calculates locus data by subjecting the calculated correction velocity data to time integration; and an index calculation device that calculates a gait index, using the locus data calculated by the locus calculation device.

A gait measurement method of one aspect of the present invention includes: detecting at least one gait phase from acceleration data measured by an inertial measurement unit; calculating velocity data by subjecting the acceleration data to time integration; calculating correction amounts associated with each of the gait phases, using the gait phases and the velocity data; calculating correction velocity data by subtracting the correction amounts from the velocity data associated with each of the gait phases; calculating locus data by subjecting the calculated correction velocity data to time integration; and calculating a gait index, using the calculated locus data.

A program of one aspect of the present invention causes a computer to perform: a process of detecting at least one gait phase from acceleration data measured by an inertial measurement unit; a process of calculating velocity data by subjecting the acceleration data to time integration; a process of calculating correction amounts associated with each of the gait phases, using the gait phases and the velocity data; a process of calculating correction velocity data by subtracting the correction amounts from the velocity data associated with each of the gait phases; a process of calculating locus data by subjecting the calculated correction velocity data to time integration; and a process of calculating a gait index, using the calculated locus data.

### Advantageous Effects of Invention

According to the present invention, it is possible to provide a gait measurement system capable of measuring gait with high accuracy.

### BRIEF DESCRIPTION OF DRAWINGS

FIG. 1 is a block diagram showing an example configuration of a gait measurement system according to a first example embodiment of the present invention.

FIG. 2 is a block diagram showing an example configuration of a locus calculation device included in the gait measurement system according to the first example embodiment of the present invention.

FIG. 3 is a conceptual diagram for explaining gait phases.

FIG. 4 is a graph for explaining acceleration data to be used in the gait measurement system according to the first example embodiment of the present invention.

FIG. 5 is a graph for explaining temporal changes in lower limb load.

FIG. 6 is a graph showing an example of velocity data calculated by the gait measurement system according to the first example embodiment of the present invention.

FIG. 7 is a graph showing an example graph obtained when the gait measurement system according to the first example embodiment of the present invention applies a first correction amount to velocity data.

FIG. 8 is a graph showing an example graph obtained when the gait measurement system according to the first example embodiment of the present invention applies a first correction amount and a second correction amount to velocity data.

FIG. 9 is a flowchart for explaining an operation of the gait measurement system according to the first example embodiment of the present invention.

FIG. 10 is a block diagram showing an example configuration of a gait measurement system according to a second example embodiment of the present invention.

FIG. 11 is a block diagram showing an example configuration of a locus calculation device included in the gait measurement system according to the second example embodiment of the present invention.

FIG. 12 is a graph for explaining acceleration data and angular velocity data to be used in the gait measurement system according to the second example embodiment of the present invention.

FIG. 13 is a graph showing an example of correction acceleration data calculated by the gait measurement system according to the second example embodiment of the present invention.

FIG. 14 is a block diagram showing an example configuration of a gait measurement system according to a modification of the second example embodiment of the present invention.

FIG. 15 is a flowchart for explaining an operation of the gait measurement system according to the first example embodiment of the present invention.

FIG. 16 is a block diagram showing an example configuration of a gait measurement system according to a third example embodiment of the present invention.

FIG. 17 is a block diagram showing an example hardware configuration in a gait measurement system according to each example embodiment of the present invention.

### EXAMPLE EMBODIMENTS

The following is a description of example embodiments of the present invention, with reference to drawings. The example embodiments described below are limited in a technically preferable manner for carrying out the present invention, but the scope of the invention is not limited to these example embodiments. In all the drawings to be used for explaining the example embodiments below, like components and aspects are denoted by like reference numerals, unless there is a particular reason. Further, in the example embodiments described below, the same configurations and operations may not be repeatedly explained.

### First Example Embodiment

First, a gait measurement system according to a first example embodiment of the present invention is described, with reference to drawings. The gait measurement system of the present example embodiment is a system that measures the gait of a user. Gait refers to the manner of walking of a human or an animal. For example, gait includes a stride length (right or left, equivalent to one step), a stride length (two steps), rhythm, velocity, dynamic basis, traveling direction, foot angle, hip angle, and ability to crouch down. In the description below, the term “walking person” refers mainly to a user who is walking, but a user who has stopped may also be referred to as a walking person.

(Configuration)

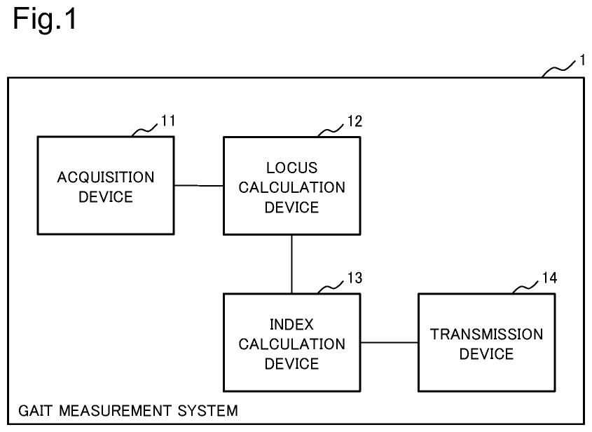

FIG. 1 is a diagram showing an example configuration of a gait measurement system 1 of the present example embodiment. As shown in FIG. 1, the gait measurement system 1 includes an acquisition device 11, a locus calculation device 12, an index calculation device 13, and a transmission device 14.

Each device may be connected to one another by cables or may be wirelessly connected, and the connection mode is not limited to any particular one. For example, each device is connected by cables such as local area network (LAN) cables or universal serial bus (USB) cables. Alternatively, each device is wirelessly connected by Bluetooth (registered trademark), Wi-Fi (registered trademark), or the like, for example.

The acquisition device 11 is attached to a body part of the user. The acquisition device 11 is connected to the locus calculation device 12. The acquisition device 11 measures acceleration, and transmits the measured acceleration to the locus calculation device 12.

The acquisition device 11 is formed with an inertial measurement unit (hereinafter referred to as IMU) that includes an accelerometer, for example. The acquisition device 11 is attached to a shoe with a clip or the like or is incorporated into the insole of a shoe, to measure accelerations in three directions. For example, the acquisition device 11 measures accelerations in the three directions: the depth direction, the vertical direction, and the horizontal direction, as viewed from the user. Alternatively, the acquisition device 11 may be attached to both feet or to only one foot. The acquisition device 11 may be designed to measure angular velocities of three axes, in addition to accelerations in the three directions. For example, the acquisition device 11 measures angular velocities in the three directions: the depth direction, the vertical direction, and the horizontal direction, as viewed from the user.

The measurement range of the accelerometer included in the acquisition device 11 preferably includes the maximum acceleration during walking of the user at the installation position of the acquisition device 11. This is because, if the measurement range of the acquisition device 11 does not cope with actions of the user, the accuracy of the locus calculation, which will be described later, drops.

The time intervals at which the acquisition device 11 measures acceleration of the user are not limited to any particular ones. However, if the time intervals between measurements are too long, there is a possibility that the accuracy of the locus calculation described later will drop. If the time intervals between measurements are too short, there is a possibility that an excessive amount of acceleration data will be transmitted. For example, the acquisition device 11 preferably measures acceleration of the user at intervals of 10 millisecond.

The locus calculation device 12 is connected to the acquisition device 11 and the index calculation device 13. The locus calculation device 12 receives the acceleration data from the acquisition device 11. The locus calculation device 12 calculates locus data, using the received acceleration data. The locus data is data indicating the locus of the attachment position of the acquisition device 11. For example, the locus data is data indicating the attachment position of the acquisition device 11 in an x-, y-, and z-coordinate system at time t. The locus calculation device 12 transmits the calculated locus data to the index calculation device 13. The specific functions and configuration of the locus calculation device 12 will be described later.

The index calculation device 13 is connected to the locus calculation device 12 and the transmission device 14. The index calculation device 13 receives the locus data from the locus calculation device 12. The index calculation device 13 calculates gait indexes from the received locus data. For example, the index calculation device 13 calculates a stride length, a gait velocity, and the like as the gait indexes. The index calculation device 13 transmits the calculated gait indexes to the transmission device 14. Specific examples of the gait indexes to be calculated by the index calculation device 13 will be described later.

The transmission device 14 receives the gait indexes from the index calculation device 13. The transmission device 14 transmits the gait indexes received from the index calculation device 13 to the outside. For example, the transmission device 14 transmits the gait indexes to a display device having a display function. Alternatively, the transmission device 14 may be designed to transmit the gait indexes to a system that manages the health condition of the user.

An example configuration of the gait measurement system 1 has been described so far. However, the configuration of the gait measurement system 1 shown in FIG. 1 is merely an example, and does not limit the configuration of the gait measurement system 1 of the present example embodiment to the same configuration as above.

[Locus Calculation Device]

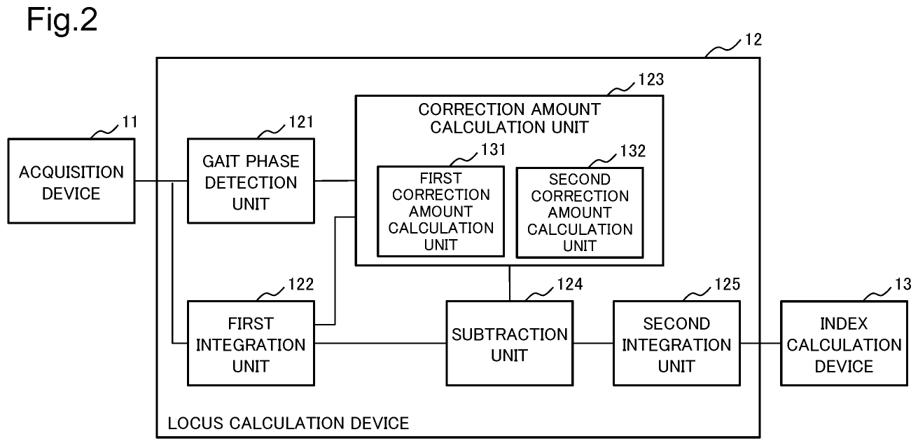

Next, an example configuration of the locus calculation device 12 is described, with reference to drawings. FIG. 2 is a block diagram showing an example configuration of the locus calculation device 12. As shown in FIG. 2, the locus calculation device 12 includes a gait phase detection unit 121, a first integration unit 122, a correction amount calculation unit 123, a subtraction unit 124, and a second integration unit 125.

As shown in FIG. 2, the gait phase detection unit 121 is connected to the acquisition device 11. The gait phase detection unit 121 is also connected to the correction amount calculation unit 123. The gait phase detection unit 121 receives acceleration data from the acquisition device 11. The gait phase detection unit 121 detects gait phases from the received acceleration data. The gait phase detection unit 121 transmits the detected gait phases to the correction amount calculation unit 123.

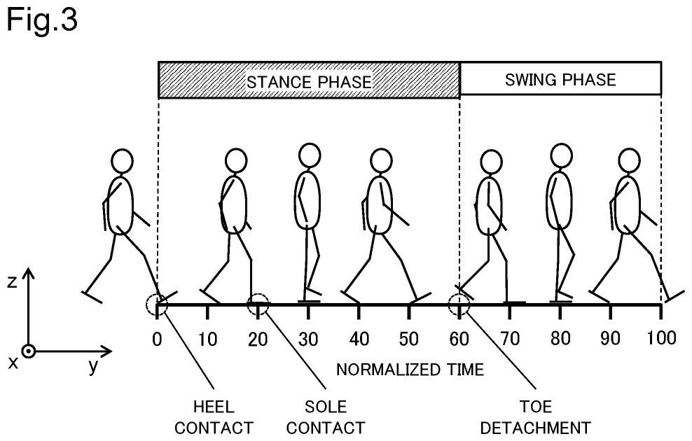

FIG. 3 is a conceptual diagram for explaining gait phases. The horizontal axis under the walking person in FIG. 3 indicates the normalized time obtained by normalizing the elapsed course associated with walking. In the description below, explanation focuses on the right foot, but the same explanation applies to the left foot. Hereinafter, as shown in FIG. 3, the lateral direction with respect to the walking person is set to the x-axis (perpendicular to the plane of the drawing sheet), the traveling direction is set to the y-axis (the lateral direction on the drawing sheet), and the vertical direction is set to the z-axis (the vertical direction on the drawing sheet).

Gait phases of a person are classified as stance phases and swing phases. A stance phase is the period that starts when the heel of one foot comes into contact with the ground (heel contact), lasts while the sole of the foot is completely in contact with the ground (sole contact), and ends when the toe of the foot leaves the ground (toe detachment). A swing phase is the period from when the toe leaves the ground until when the heel next comes into contact with the ground. A stance phase and the swing phase that follows are called one gait cycle.

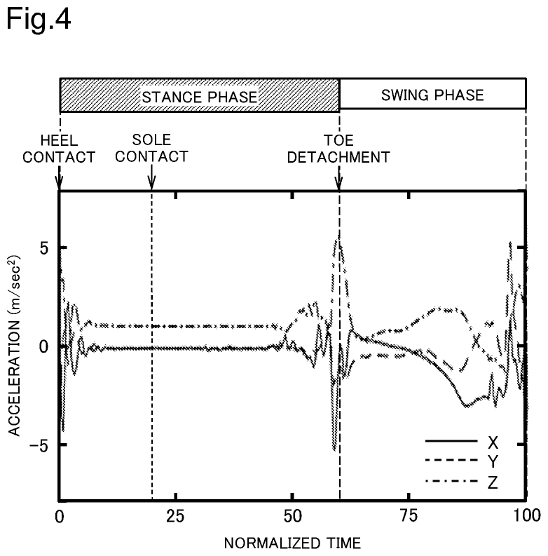

FIG. 4 is an example of data showing temporal changes in acceleration measured by the acquisition device 11 attached to the toe portion of the right foot in one gait cycle (this data will be hereinafter also referred to as “acceleration waveforms”). As can be seen from FIG. 4, a high acceleration occurs at the moment of heel contact and at the moment of toe detachment.

The gait phase detection unit 121 performs peak detection on the acceleration waveforms. In a case where the absolute value of the highest detected peak is equal to or greater than a certain value, the gait phase detection unit 121 determines that the time at which the highest peak has been detected is the moment of heel contact or toe detachment (the time will be hereinafter referred to as the “maximum peak time”). As can be seen from the example shown in FIG. 4, heel contact and toe detachment can be detected when the threshold for the absolute value of the highest peak of acceleration waveforms is set at about 4.5 meters per second squared (m/sect) or greater.

The gait phase detection unit 121 then compares a variation V1 in acceleration during a period from the maximum peak time until the time before a predetermined period, with a variation V2 in acceleration during a period from the maximum peak time till the time after the predetermined period. Since the time before heel contact is in a swing phase, the variation in acceleration increases due to movement of the foot. On the other hand, the time after heel contact is in a stance phase, and therefore, the foot does not move, generating no variation in acceleration. Accordingly, if the variation V1 is larger than the variation V2, the gait phase detection unit 121 determines that the maximum peak time is a heel contact time. If the variation V1 is smaller than the variation V2, the gait phase detection unit 121 determines that the maximum peak time is a toe detachment time.

Here, the time of heel contact in the nth gait cycle is defined as t_{1C}(n), the time of toe detachment is defined as t_{TO}(n), and the representative time of sole contact is defined as t_{FF}(n). In this case, the representative time t_{FF}(n) of sole contact is expressed by Expression 1 shown below.

t_{FF}(n)=0.2×[t_{1C}(n+1)−t_{1C}(n)]+t_{1c}(n) (1)

The representative time t_{FF}(n) of sole contact defined by Expression 1 is the time point of 20% of the gait cycle. This time point corresponds to the mid stance under the classification of motion analysis. The representative time t_{FF}(n) of sole contact may be defined differently from Expression 1. For example, the representative time t_{FF}(n) of sole contact may be defined by Expression 2 shown below.

$${t}_{F F} ( n ) = \frac{{t}_{T O} ( n ) + {t}_{I C} ( n )}{2} \qquad ( 2 )$$

The representative time t_{FF}(n) of sole contact defined by Expression 2 is the middle of the stance phase. In this manner, the representative time t_{FF}(n) of sole contact can be set at any appropriate time during the sole contact period.

The gait phase detection unit 121 determines the period from heel contact to toe detachment as a stance phase, and determines the period from toe detachment to heel contact as a swing phase.

In the above manner, the gait phase detection unit 121 can detect gait phases that are a stance phase and a swing phase. By the method described above, gait phases are formed with a stance phase and a swing phase, and the stance phase and the swing phase are detected. However, a gait phase detection method to be implemented by the gait phase detection unit 121 is not limited to the technique described above.

As shown in FIG. 2, the first integration unit 122 is connected to the acquisition device 11. The first integration unit 122 is also connected to the correction amount calculation unit 123 and the subtraction unit 124. The first integration unit 122 receives acceleration data from the acquisition device 11. The first integration unit 122 performs time integration on the received acceleration data, to calculate velocity data. The first integration unit 122 transmits the calculated velocity data to the correction amount calculation unit 123 and the subtraction unit 124.

The first integration unit 122 calculates velocity data v(t) by calculating the sum of the values obtained by dividing the acceleration data a(t) by a sampling frequency Fₛ during the period from the time t_{1C}(n) of heel contact to the time t, according to Expression 3 shown below.

$$v ( t ) = {\sum }_{i = {t}_{IC} ( n )}^{t} \frac{a ( i )}{{f}_{s}} \qquad ( 3 )$$

In Expression 3, the acceleration data a(t) is the vector indicating the respective accelerations in the X-, Y-, and Z-axis directions, and is expressed by Expression 4 shown below. Also, in Expression 3, the velocity data v(t) is the vector indicating the respective velocities in the X-, Y-, and Z-axis directions, and is expressed by Expression 5 shown below.

a(t)=[aₓ(t),a_{y}(t),a_{Z}(t)]^{T} (4)

v(t)=[vₓ(t),v_{y}(t),v_{z}(t)]^{T} (5)

As shown in FIG. 2, the correction amount calculation unit 123 is connected to the gait phase detection unit 121, the first integration unit 122, and the subtraction unit 124. As shown in FIG. 2, the correction amount calculation unit 123 includes a first correction amount calculation unit 131 and a second correction amount calculation unit 132. The correction amount calculation unit 123 calculates correction amounts, using the data received from the gait phase detection unit 121 and the first integration unit 122. The correction amount calculation unit 123 transmits the calculated correction amounts to the subtraction unit 124.

The first correction amount calculation unit 131 receives the gait phases of the nth gait cycle from the gait phase detection unit 121. The correction amount calculation unit 123 calculates a first correction amount in the nth gait cycle, using the received gait phases. The first correction amount is a correction amount for making the velocity bias in the stance phase zero, taking the initial velocity into account. The first correction amount calculation unit 131 outputs the calculated first correction amount to the subtraction unit 124.

The first correction amount calculation unit 131 calculates the first correction amount, using a constant C expressed by Expression 6 shown below.

C=−v[t_{FF}(n)] (6)

The second correction amount calculation unit 132 receives the velocity data of the nth gait cycle from the first integration unit 122. The second correction amount calculation unit 132 calculates a second correction amount in the nth gait cycle, using the received velocity data. The second correction amount is a correction amount for reducing drift in the swing phase, and performing correction so that the velocity at the time of sole contact becomes zero. The second correction amount calculation unit 132 outputs the calculated second correction amount to the subtraction unit 124.

To calculate the second correction amount, the second correction amount calculation unit 132 uses a function f(t) related to time t expressed by Expression 7 shown below. In Expression 7, the function f(t) is a correction amount defined in the swing phase, which is from time t_{TO}(n) to t_{1C}(n+1), and is set to zero in the stance phase.

$$f ( t ) = \{ \begin{matrix}0 & ( {t}_{I C} ( n ) \leq t < {t}_{T O} ( n ) ) \\ \frac{- ( t - {t}_{T O} ( n ) )}{{t}_{F F} ( n + 1 ) - {t}_{T O} ( n )} \times [ v ( {t}_{F F} ( n + 1 ) ) - v ( {t}_{F F} ( n ) ) ] & ( {t}_{T O} ( n ) \leq t < {t}_{I C} ( n + 1 ) )\end{matrix} \qquad ( 7 )$$

As shown in FIG. 2, the subtraction unit 124 is connected to the first integration unit 122, the correction amount calculation unit 123, and the second integration unit 125. The subtraction unit 124 receives the velocity data from the first integration unit 122. The subtraction unit 124 also receives the first correction amount and the second correction amount from the correction amount calculation unit 123. The subtraction unit 124 subtracts the first correction amount and the second correction amount from the received velocity data, to calculate correction velocity data. The subtraction unit 124 transmits the calculated correction velocity data to the second integration unit 125.

The subtraction unit 124 calculates correction velocity data v_{c}(t), using Expression 8 shown below.

v_{c}(t)=v(t)−C−f(t) (8)

As described above, as the function f(t) is defined in the swing phase and is set to zero in the stance phase, different correction amounts are applied to the gait phases that are the stance phase and the swing phase.

As shown in FIG. 2, the second integration unit 125 is connected to the subtraction unit 124. The second integration unit 125 is also connected to the index calculation device 13. The second integration unit 125 receives the correction velocity data v_{c}(t) from the subtraction unit 124. The second integration unit 125 performs time integration on the received correction velocity data v_{c}(t), to calculate locus data x(t). The second integration unit 125 transmits the calculated locus data x(t) to the index calculation device 13.

The second integration unit 125 calculates the locus data x(t) by calculating the sum of the values obtained by dividing the correction velocity data v_{c}(t) by the sampling frequency fₛ during the period from the time t_{1C}(n) of heel contact to the time t, according to Expression 9 shown below.

$$x ( t ) = {\sum }_{i = {t}_{IC} ( n )}^{t} \frac{{v}_{c} ( i )}{{f}_{s}} ( {t}_{IC} ( n ) \leq t < {t}_{IC} ( n + 1 ) ) \qquad ( 9 )$$

In Expression 9, the locus data x(t) is the x-, y-, and z-coordinates of the position of the acquisition device 11 at time t, and is expressed by Expression 10 shown below. For example, in a case where the acquisition device 11 is attached to the toe, the locus data x(t) indicates the position of the toe.

x(t)=[xₓ(t),x_{y}(t),x_{z}(t)]^{T} (10)

An example configuration of the locus calculation device 12 has been described so far. However, the configuration of the locus calculation device 12 shown in FIG. 2 is merely an example, and does not limit the configuration of the locus calculation device 12 of the present example embodiment to the same configuration as above.

Next, an example in which the index calculation device 13 calculates gait indexes by using the locus data x(t) calculated by the locus calculation device 12 is described.

The index calculation device 13 receives the locus data x(t) from the locus calculation device 12. Using the received locus data x(t), the index calculation device 13 calculates gait indexes such as a stride length L and a gait velocity v as numerical values for quantitatively evaluating gait of a person.

For example, the index calculation device 13 calculates the stride length L, using Expression 11 shown below. The numerator in Expression 11 is called an overlapped step distance, and is almost twice the stride length. The overlapped step distance indicates the amount of movement of the foot from heel contact to the next heel contact, and is also called a stride distance or a stride length.

$$L = \frac{\vert x ( {t}_{IC} ( n + 1 ) ) - x ( {t}_{IC} ( n ) ) \vert}{2} \qquad ( 11 )$$

The index calculation device 13 also calculates the gait velocity v as a gait index, using Expression 12 shown below, for example.

$$v = \frac{2 L}{{t}_{IC} ( n + 1 ) - {t}_{IC} ( n )} \qquad ( 12 )$$

The gait indexes to be calculated by the index calculation device 13 are not limited to a stride length and a gait velocity. For example, the index calculation device 13 may calculate a “toe clearance” that indicates the minimum distance between the toe and the ground in the swing phase and is known to be correlated with the risk of fall, as a gait index. Alternatively, for example, the index calculation device 13 may calculate, as a gait index, a foot lateral movement amount indicating the degree of circumduction gait that is observed in a stroke paralysis patient.

The above is a description of an example in which the index calculation device 13 calculates gait indexes by using the locus data x(t) calculated by the locus calculation device 12.

[Correction Amounts]

Velocity data correction using the first correction amount and the second correction amount is now described, with reference to drawings.

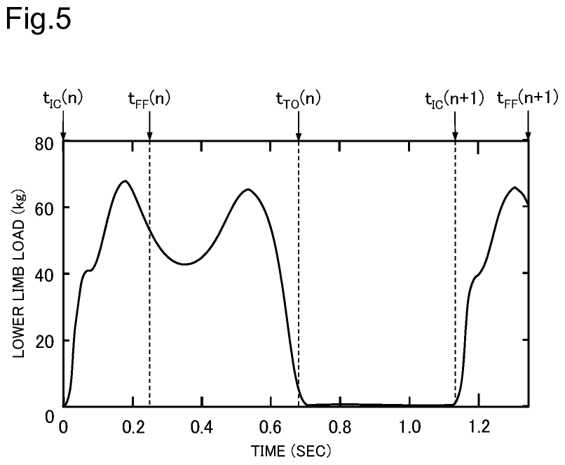

FIG. 5 is a graph showing temporal changes in the reference value of the lower limb load during walking. Here, t_{1C}(n), t_{FF}(n), t_{TO}(n), t_{1C}(n+1), and t_{FF}(n+1) are times related to the gait phases calculated by the gait phase detection unit 121. FIG. 5 shows a situation where a stance phase in which the body weight is supported starts as a result of an increase in the load at the heel contact time t_{1C}(n), the load gradually decreases as the swing phase draws near, and the load becomes zero at the toe detachment time t_{TO}(n).

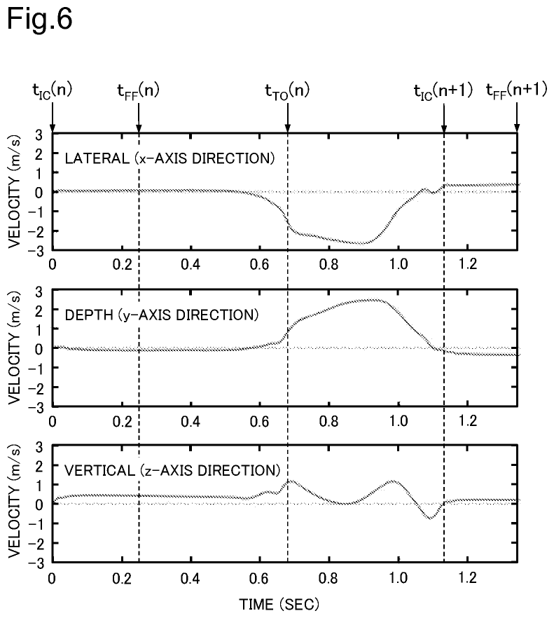

FIG. 6 shows an example of the velocity data calculated by the first integration unit 122. FIG. 6 shows an example of waveforms indicating temporal changes in velocities in the directions of the x-axis (lateral), the y-axis (depth), and the z-axis (vertical), in this order from the top (the waveforms will be hereinafter also referred to as velocity waveforms). The velocity data in FIG. 6 is not obtained from the acceleration data shown in FIG. 4.

In the velocity waveform in the z-axis direction in FIG. 6, the velocity at the representative time t_{FF}(n) of sole contact indicates a positive value, though the velocity should be zero at the time of sole contact because the foot is secured unless sideslip occurs. This is an error caused by the fact that the initial velocity (the velocity at the moment of heel contact) was assumed to be zero in the integration according to Expression 3. In reality, however, the toe moved vertically downward due to plantar flexion even after heel contact, and the initial velocity was not zero.

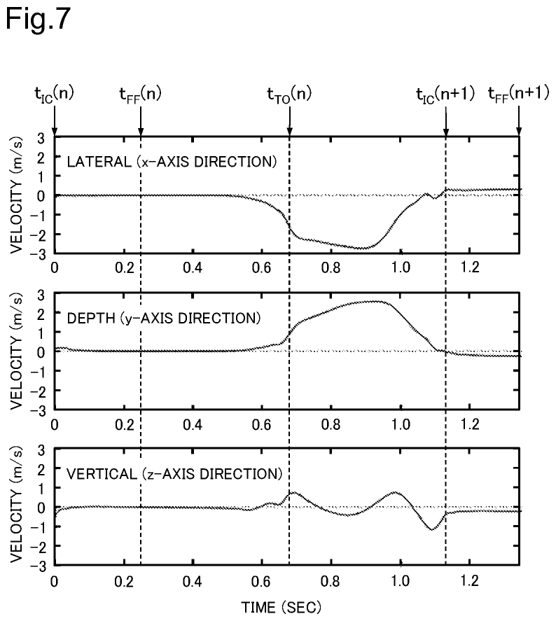

FIG. 7 shows first correction velocity data v_{c1}(t) obtained when the first correction amount is applied to velocity data calculated by the first integration unit 122. The first correction velocity data v_{c1}(t) is calculated according to Expression 13 shown below.

v_{c1}(t)=v(t)−C (13)

The first correction amount is a correction amount for making the velocity bias in the stance phase zero, considering the initial velocity. As can be seen from the velocities in each axis direction at the representative time t_{FF}(n) of sole contact in FIG. 7, the velocity bias turns to zero, and appropriately copes with the state in which the foot is secured at the time of sole contact.

In FIG. 7, however, the velocity at the representative time t_{FF}(n+1) of sole contact in the next gait cycle is not zero. This is a drift error caused by integration of accumulation of measurement errors due to changes in the angle of the sensor in the swing phase. This drift error has characteristics different from those of an error in the stance phase, and therefore, the drift error cannot be corrected with the first correction amount.

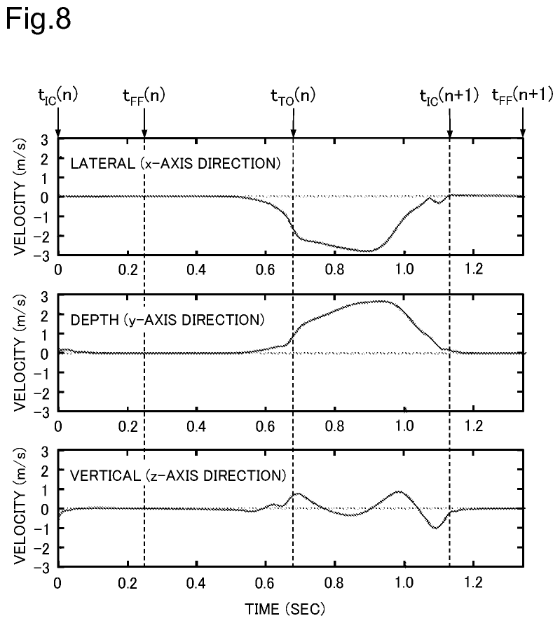

FIG. 8 shows second correction velocity data obtained by further applying the second correction amount to the velocity data (first correction velocity data) corrected with the first correction amount. In the second correction velocity data, the drift error in the swing phase is eliminated, and the velocity at the representative time t_{FF}(n+1) of sole contact in the next gait cycle is corrected to be zero.

As velocity data is corrected with the first correction amount and the second correction amount as described above, the velocity bias in the stance phase can be made zero, and the drift error in the swing phase can be eliminated.

The configuration of the gait measurement system 1 of the present example embodiment has been described so far. However, the configuration of the gait measurement system 1 described above with reference to FIGS. 1 to 8 is merely an example, and does not necessarily limit the configuration of the gait measurement system 1 of the present example embodiment to the same configuration as above.

(Operation)

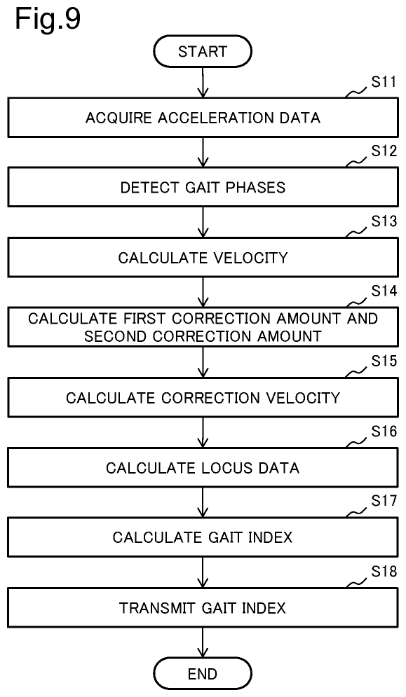

Next, an operation of the gait measurement system 1 of the present example embodiment is described, with reference to a drawing. FIG. 9 is a flowchart for explaining an operation of the gait measurement system 1. In the description with reference to the flowchart shown in FIG. 9, each of the components constituting the gait measurement system 1 will be described as a principal operator. However, the principal operator in the process according to the flowchart in FIG. 9 may be regarded as the gait measurement system 1.

In FIG. 9, the acquisition device 11 first acquires acceleration data (step S11).

The gait phase detection unit 121 of the locus calculation device 12 then detects gait phases from the acceleration data (step S12).

The first integration unit 122 of the locus calculation device 12 then performs time integration on the acceleration data, to calculate velocity data (step S13).

The correction amount calculation unit 123 of the locus calculation device 12 then calculates a first correction amount and a second correction amount, using the gait phases and the velocity data (step S14).

The subtraction unit 124 of the locus calculation device 12 then subtracts the first correction amount and the second correction amount from the velocity data, to calculate correction velocity data (step S15).

The second integration unit 125 of the locus calculation device 12 then performs time integration on the correction velocity data, to calculate locus data (step S16).

The index calculation device 13 then calculates gait indexes from the locus data (step S17).

The transmission device 14 then transmits the gait indexes to a display device (step S18).

An operation of the gait measurement system 1 of the present example embodiment has been described so far. However, the operation of the gait measurement system 1 according to the flowchart in FIG. 9 is merely an example, and does not limit operations of the gait measurement system 1 of the present example embodiment to the same operation as above.

As described above, a gait measurement system of the present example embodiment includes a locus calculation device and an index calculation device. The locus calculation device detects at least one gait phase from acceleration data measured by an inertial measurement unit, and calculates velocity data by subjecting the acceleration data to time integration. The locus calculation device calculates correction amounts associated with each of the gait phases by using the gait phases and the velocity data, and calculates correction velocity data by subtracting the correction amounts from the velocity data associated with each of the gait phases. The locus calculation device calculates locus data by subjecting the calculated correction velocity data to time integration. The index calculation device calculates gait indexes, using the locus data calculated by the locus calculation device.

The locus calculation device of the present example embodiment includes a gait phase detection unit, a first integration unit, a correction amount calculation unit, a subtraction unit, and a second integration unit. The gait phase detection unit acquires acceleration data from the inertial measurement unit, and detects at least one gait phase, using the acquired acceleration data. The first integration unit acquires the acceleration data from the inertial measurement unit, and calculates velocity data by subjecting the acquired acceleration data to time integration. The correction amount calculation unit calculates correction amounts associated with each of the gait phases, using the gait phases and the velocity data. The subtraction unit subtracts the correction amounts associated with each of the gait phases from the velocity data, to calculate correction velocity data. The second integration unit subjects the correction velocity data to time integration, to calculate locus data.

The gait phase detection unit of the present example embodiment detects a stance phase and a swing phase as gait phases. The correction amount calculation unit calculates a first correction amount for making the velocity bias zero in the stance phase and a second correction amount for reducing drift in the swing phase, and calculates correction velocity data by subtracting the calculated first correction amount and second correction amount from the velocity data. For example, the gait phase detection unit detects a period from heel contact to toe detachment as a stance phase, and detects a period from toe detachment to heel contact as a swing phase.

With the gait measurement system of the present example embodiment, it is possible to eliminate drift that shows different characteristics between a stance phase and a swing phase, and thus, gait can be measured with high accuracy.

### Second Example Embodiment

Next, a gait measurement system according to a second example embodiment of the present invention is described, with reference to drawings. The gait measurement system of the present example embodiment differs from the gait measurement system of the first example embodiment in that angular velocity data is used for coordinate transform of acceleration data.

(Configuration)

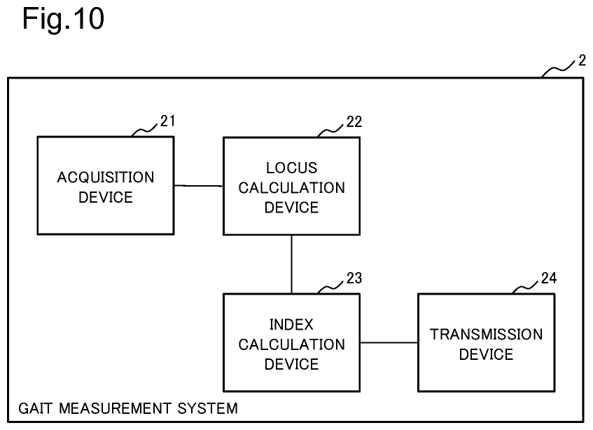

FIG. 10 is a diagram showing an example configuration of a gait measurement system 2 of the present example embodiment. As shown in FIG. 10, the gait measurement system 2 includes an acquisition device 21, a locus calculation device 22, an index calculation device 23, and a transmission device 24. In the description below, explanation of the same aspects as those of the gait measurement system 1 of the first example embodiment may not be made.

The acquisition device 21 is attached to a body part of the user. The acquisition device 21 is connected to the locus calculation device 22. The acquisition device 21 measures acceleration and angular velocity, and transmits the measured acceleration and angular velocity to the locus calculation device 22.

The acquisition device 21 is formed with an IMU that includes an accelerometer and an angular velocity meter, for example. The acquisition device 21 is attached to a shoe with a clip or the like or is incorporated into the insole of a shoe, to measure accelerations in three directions and angular velocities of three axes, for example. Alternatively, the acquisition device 11 may be attached to both feet or to only one foot.

The measurement range of the accelerometer included in the acquisition device 21 preferably includes the maximum acceleration during walking of the user at the installation position of the acquisition device 21. Likewise, the measurement range of the angular velocity meter included in the acquisition device 21 preferably includes the maximum angular velocity during walking of the user at the installation position of the acquisition device 21. This is because, if the measurement range of the acquisition device 21 does not cope with actions of the user, the accuracy of the locus calculation, which will be described later, drops. The time intervals at which the acquisition device 21 measures acceleration and angular velocity of the user are not limited to any particular ones.

The locus calculation device 22 is connected to the acquisition device 21 and the index calculation device 23. The locus calculation device 22 receives the acceleration data and the angular velocity data from the acquisition device 21. Using the angular velocity data, the acquisition device 21 performs coordinate transform to transform the coordinate system of the acceleration data into a world coordinate system, and thus, generates correction acceleration data. The locus calculation device 22 calculates locus data, using the correction acceleration data subjected to the coordinate transform. The locus calculation device 22 transmits the calculated locus data to the index calculation device 23. The specific functions and configuration of the locus calculation device 22 will be described later.

The index calculation device 23 is connected to the locus calculation device 22 and the transmission device 24. The index calculation device 23 receives the locus data from the locus calculation device 22. The index calculation device 23 calculates gait indexes from the received locus data. For example, the index calculation device 23 calculates a stride length, a gait velocity, and the like as the gait indexes. The index calculation device 23 transmits the calculated gait indexes to the transmission device 24.

The transmission device 24 receives the gait indexes from the index calculation device 23. The transmission device 24 transmits the gait indexes received from the index calculation device 23 to the outside.

An example configuration of the gait measurement system 2 has been described so far. However, the configuration of the gait measurement system 2 shown in FIG. 10 is merely an example, and does not limit the configuration of the gait measurement system 2 of the present example embodiment to the same configuration as above.

[Locus Calculation Device]

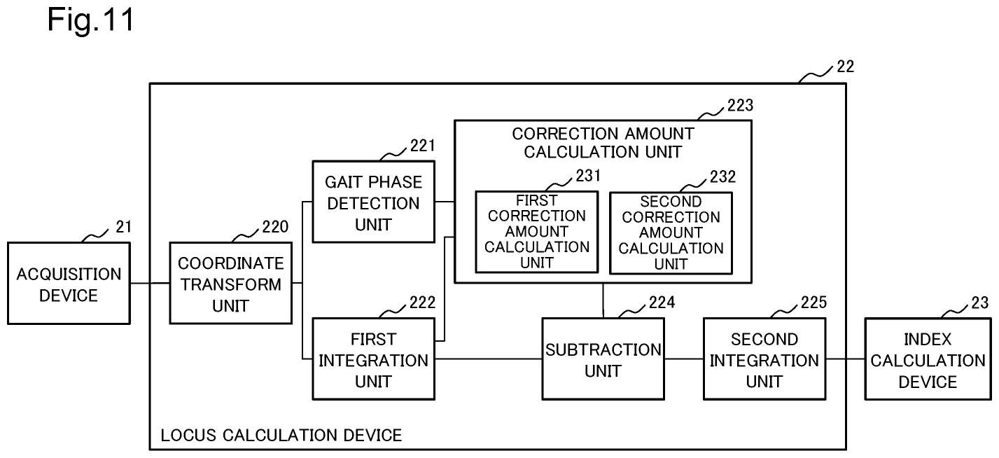

Next, an example configuration of the locus calculation device 22 is described, with reference to drawings. FIG. 11 is a block diagram showing an example configuration of the locus calculation device 22. As shown in FIG. 11, the locus calculation device 22 includes a coordinate transform unit 220, a gait phase detection unit 221, a first integration unit 222, a correction amount calculation unit 223, a subtraction unit 224, and a second integration unit 225.

As shown in FIG. 11, the coordinate transform unit 220 is connected to the acquisition device 21. The coordinate transform unit 220 is also connected to the gait phase detection unit 221 and the first integration unit 222. The coordinate transform unit 220 acquires acceleration data and angular velocity data from the acquisition device 21. Using the angular velocity data, the coordinate transform unit 220 performs coordinate transform to transform the coordinate system of the acceleration data into a world coordinate system, and thus, generates correction acceleration data. The correction acceleration data generated by the coordinate transform unit 220 is equivalent to acceleration data that is generated with the posture of the IMU with respect to the world coordinate system being taken into account. The coordinate transform unit 220 transmits the correction acceleration data to the gait phase detection unit 221 and the first integration unit 222.

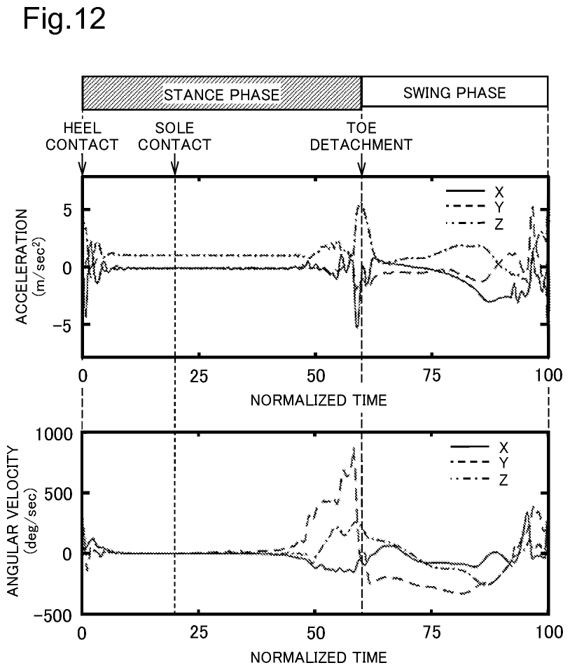

FIG. 12 is an example of data showing temporal changes in acceleration (this data will be hereinafter also referred to as “acceleration waveforms”) and data showing temporal changes in angular velocity (this data will be hereinafter also referred to as “angular velocity waveforms”), the acceleration and the angular velocity being measured by the acquisition device 11 attached to the toe portion of the right foot in one gait cycle. As can be seen from FIG. 12, a high acceleration and a high angular velocity occur at the moment of heel contact and at the moment of toe detachment.

For example, the coordinate transform unit 220 corrects the acceleration data, using a technique of Madgwick disclosed in NPL 1 shown below or the like.
NPL 1: S. Madgwick, A. Harrison, and R. Vaidyanathan, “Estimation of IMU and MARG orientation using a gradient descent algorithm”, 2011 IEEE International Conference on Rehabilitation Robotics, Rehab Week Zurich, ETH Zurich Science City, Switzerland, Jun. 29-Jul. 1, 2011, pp. 179-185.

The general approach is to calculate the posture of the IMU, using the integral of angular velocity. However, angular velocity measurement data has errors mainly due to bias, and the errors are accumulated by integration. The Madgwick's technique reduces error accumulation by integrally using angular velocity measurement data and acceleration measurement data, based on gravitational acceleration.

For example, the coordinate transform unit 220 performs coordinate transform, to transform the acceleration data a(t) in the IMU coordinate system (also referred to as a solid coordinate system) expressed by Expression 4 shown above, into correction acceleration data a_{c}(t) in a world coordinate system according to Expression 14 shown below. Here, R⁻¹(t) represents the inverse of a three-dimensional rotation matrix R(t).

a_{c}(t)=R⁻¹(t)·a(t) (14)

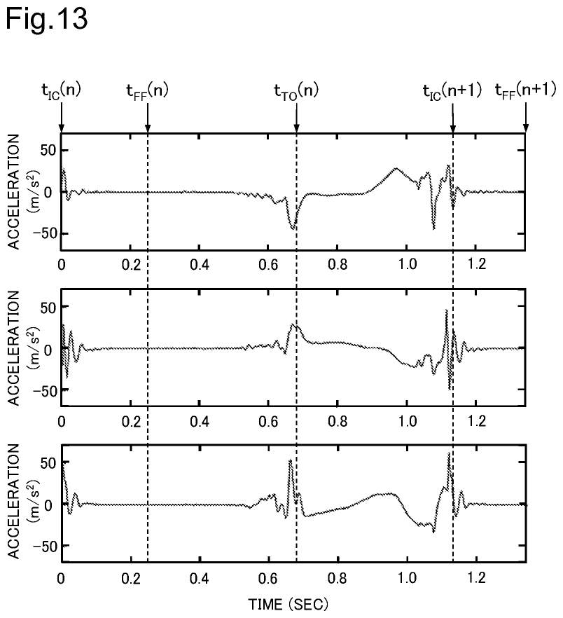

FIG. 13 shows an example of the correction acceleration data to be generated by the coordinate transform unit 220. The graph in FIG. 13 shows an example of waveforms indicating temporal changes in correction accelerations in the directions of the x-axis (lateral), the y-axis (depth), and the z-axis (vertical), in this order from the top (the waveforms will be hereinafter also referred to as correction acceleration waveforms). The correction acceleration data in FIG. 13 is not obtained from the acceleration data and the angular velocities shown in FIG. 12.

The gait phase detection unit 221 is connected to the coordinate transform unit 220 and the correction amount calculation unit 223. The gait phase detection unit 221 receives the correction acceleration data from the coordinate transform unit 220. The gait phase detection unit 221 detects gait phases from the received correction acceleration data. The gait phase detection unit 221 transmits the detected gait phases to the correction amount calculation unit 223.

The gait phase detection unit 221 performs peak detection on the acceleration waveforms. In a case where the absolute value of the highest detected peak is equal to or greater than a certain value, the gait phase detection unit 221 determines that the time at which the highest peak has been detected is the moment of heel contact or toe detachment (the time will be hereinafter referred to as the “maximum peak time”).

The gait phase detection unit 221 then compares a variation V1 in acceleration during a period from the maximum peak time till the time before a predetermined period, with a variation V2 in acceleration during a period from the maximum peak time till the time after the predetermined period. Since the time before heel contact is in a swing phase, the variation in acceleration increases due to movement of the foot. On the other hand, the time after heel contact is in a stance phase, and therefore, the foot does not move, generating no variation in acceleration. Accordingly, if the variation V1 is larger than the variation V2, the gait phase detection unit 221 determines that the maximum peak time is a heel contact time. If the variation V1 is smaller than the variation V2, the gait phase detection unit 221 determines that the maximum peak time is a toe detachment time.

The gait phase detection unit 221 determines the period from heel contact to toe detachment as a stance phase, and determines the period from toe detachment to heel contact as a swing phase.

In the above manner, the gait phase detection unit 221 can detect gait phases that are a stance phase and a swing phase. By the method described above, gait phases are formed with a stance phase and a swing phase, and the stance phase and the swing phase are detected. However, a gait phase detection method to be implemented by the gait phase detection unit 221 is not limited to the technique described above.

The first integration unit 222 is connected to the coordinate transform unit 220, the correction amount calculation unit 223, and the subtraction unit 224. The first integration unit 222 receives the correction acceleration data from the coordinate transform unit 220. The first integration unit 222 performs time integration on the received acceleration data, to calculate velocity data. The first integration unit 222 transmits the calculated velocity data to the correction amount calculation unit 223 and the subtraction unit 224.

The correction amount calculation unit 223 is connected to the gait phase detection unit 221, the first integration unit 222, and the subtraction unit 224. As shown in FIG. 11, the correction amount calculation unit 223 includes a first correction amount calculation unit 231 and a second correction amount calculation unit 232. The correction amount calculation unit 223 calculates correction amounts, using the data received from the gait phase detection unit 221 and the first integration unit 222. The correction amount calculation unit 223 transmits the calculated correction amounts to the subtraction unit 224.

The first correction amount calculation unit 231 receives the gait phases of the nth gait cycle from the gait phase detection unit 221. The correction amount calculation unit 223 calculates a first correction amount in the nth gait cycle, using the received gait phases. The first correction amount is a correction amount for making the velocity bias in the stance phase zero, taking the initial velocity into account. The first correction amount calculation unit 231 outputs the calculated first correction amount to the subtraction unit 224.

The second correction amount calculation unit 232 receives the velocity data of the nth gait cycle from the first integration unit 222. The second correction amount calculation unit 232 calculates a second correction amount in the nth gait cycle, using the received velocity data. The second correction amount is a correction amount for reducing drift in the swing phase, and performing correction so that the velocity at the time of sole contact becomes zero. The second correction amount calculation unit 232 outputs the calculated second correction amount to the subtraction unit 224.

The subtraction unit 224 is connected to the first integration unit 222, the correction amount calculation unit 223, and the second integration unit 225. The subtraction unit 224 receives the velocity data from the first integration unit 222. The subtraction unit 224 also receives the first correction amount and the second correction amount from the correction amount calculation unit 223. The subtraction unit 224 subtracts the first correction amount and the second correction amount from the received velocity data, to calculate correction velocity data. The subtraction unit 124 transmits the calculated correction velocity data to the second integration unit 225.

The second integration unit 225 is connected to the subtraction unit 224. The second integration unit 225 is also connected to the index calculation device 23. The second integration unit 225 receives the correction velocity data v_{c}(t) from the subtraction unit 224. The second integration unit 225 performs time integration on the received correction velocity data v_{c}(t), to calculate locus data x(t). The second integration unit 225 transmits the calculated locus data x(t) to the index calculation device 23.

An example configuration of the locus calculation device 22 has been described so far. However, the configuration of the locus calculation device 22 shown in FIG. 11 is merely an example, and does not limit the configuration of the locus calculation device 22 of the present example embodiment to the same configuration as above.

The index calculation device 23 receives the locus data x(t) from the locus calculation device 22. Using the received locus data x(t), the index calculation device 23 calculates gait indexes such as a stride length L and a gait velocity v as numerical values for quantitatively evaluating gait of a person.

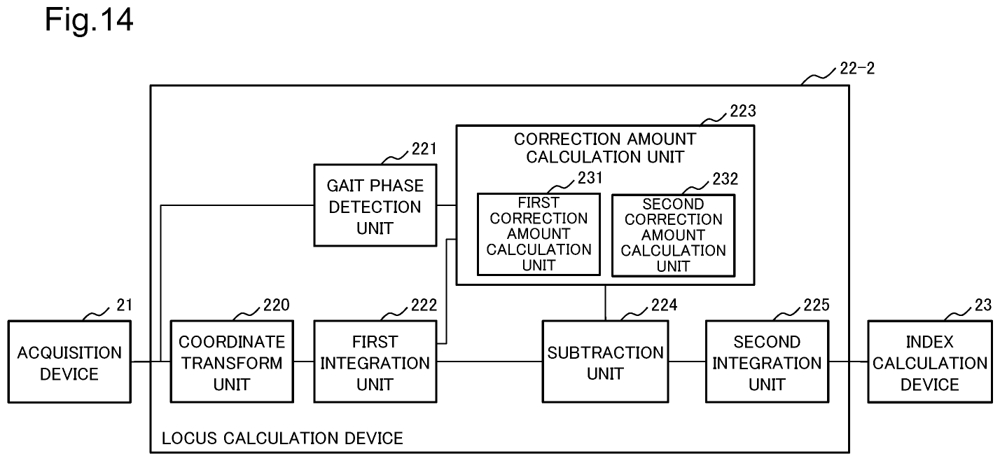

A modification of the locus calculation device 22 is now described, with reference to a drawing. FIG. 14 is a block diagram showing an example configuration of a locus calculation device 22-2 according to the modification. The locus calculation device 22-2 shown in FIG. 14 differs from the locus calculation device 22 shown in FIG. 11 in that the gait phase detection unit 221 acquires acceleration data from the acquisition device 21 and detects gait phases using the acceleration data. The other aspects of the locus calculation device 22-2 are the same as those of the locus calculation device 22, and therefore, detailed explanation of them is not made herein.

The configuration of the gait measurement system 1 of the present example embodiment has been described so far. However, the configuration of the gait measurement system 2 described above with reference to FIGS. 10 and 11 is merely an example, and does not necessarily limit the configuration of the gait measurement system 2 of the present example embodiment to the same configuration as above.

(Operation)

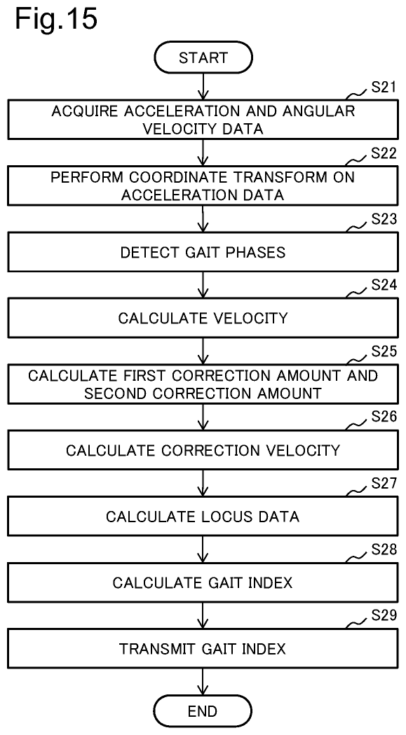

Next, an operation of the gait measurement system 2 of the present example embodiment is described, with reference to a drawing. FIG. 15 is a flowchart for explaining an operation of the gait measurement system 2. In the description with reference to the flowchart shown in FIG. 15, each of the components constituting the gait measurement system 2 will be described as a principal operator. However, the principal operator in the process according to the flowchart in FIG. 15 may be regarded as the gait measurement system 2.

In FIG. 15, the acquisition device 21 acquires acceleration data and angular velocity data (step S21).

The coordinate transform unit 20 then calculates correction acceleration data, using the acceleration data and the angular velocity data (step S22).

The gait phase detection unit 121 of the locus calculation device 22 then detects gait phases from the correction acceleration data (step S23).

The first integration unit 222 of the locus calculation device 22 then performs time integration on the correction acceleration data, to calculate velocity data (step S24).

The correction amount calculation unit 223 of the locus calculation device 22 then calculates a first correction amount and a second correction amount, using the gait phases and the velocity data (step S25).

The subtraction unit 224 of the locus calculation device 22 then subtracts the first correction amount and the second correction amount from the velocity data, to calculate correction velocity data (step S26).

The second integration unit 225 of the locus calculation device 22 then performs time integration on the correction velocity data, to calculate locus data (step S27).

The index calculation device 23 then calculates gait indexes from the locus data (step S28).

The transmission device 24 then transmits the gait indexes to a display device (step S29).

An operation of the gait measurement system 2 of the present example embodiment has been described so far. However, the operation of the gait measurement system 2 according to the flowchart in FIG. 15 is merely an example, and does not limit operations of the gait measurement system 2 of the present example embodiment to the same operation as above.

As described above, a locus calculation device of a gait measurement system of the present example embodiment includes a coordinate transform unit, in addition to a gait phase detection unit, a first integration unit, a correction amount calculation unit, a subtraction unit, and a second integration unit. The coordinate transform unit acquires acceleration data and angular velocity data from an inertial measurement unit, and performs coordinate transform to transform the acceleration data into correction acceleration data in a world coordinate system, using the acquired angular velocity data. The gait phase detection unit acquires the correction acceleration data from the coordinate transform unit, and detects at least one gait phase, using the acquired correction acceleration data. The first integration unit acquires the correction acceleration data from the coordinate transform unit, and calculates velocity data by subjecting the acquired correction acceleration data to time integration. The correction amount calculation unit calculates correction amounts associated with each of the gait phases, using the gait phases and the velocity data. The subtraction unit subtracts the correction amounts associated with each of the gait phases from the velocity data, to calculate correction velocity data. The second integration unit subjects the correction velocity data to time integration, to calculate locus data.

In the locus calculation device of another mode of the gait measurement system of the present example embodiment, the coordinate transform unit acquires acceleration data and angular velocity data from an inertial measurement unit, and performs coordinate transform to transform the acceleration data into correction acceleration data in a world coordinate system, using the acquired angular velocity data. The gait phase detection unit acquires acceleration data from the inertial measurement unit, and detects at least one gait phase, using the acquired correction acceleration data. The first integration unit acquires the correction acceleration data from the coordinate transform unit, and calculates velocity data by subjecting the acquired correction acceleration data to time integration. The correction amount calculation unit calculates correction amounts associated with each of the gait phases, using the gait phases and the velocity data. The subtraction unit subtracts the correction amounts associated with each of the gait phases from the velocity data, to calculate correction velocity data. The second integration unit subjects the correction velocity data to time integration, to calculate locus data.

For example, the coordinate transform unit of the gait measurement system of the present example embodiment performs coordinate transformation to transform acceleration data into correction acceleration data in a world coordinate system, using the technique of Madgwick.

In the gait measurement system of the present example embodiment, the coordinate transform unit detects changes in the posture of the IMU with respect to a world coordinate system, and corrects acceleration data, to eliminate the influence of the changes in the posture of the IMU. Thus, with the gait measurement system of the present example embodiment, the posture of the IMU to be attached can be determined as desired. Further, with the gait measurement system of the present example embodiment, the gait calculation accuracy in a case where the posture of the IMU changes due to bottom dorsiflexion of the walking person or dislocation of the sensor during walking becomes higher.

### Third Example Embodiment

Next, a gait measurement system according to a third example embodiment of the present invention is described, with reference to a drawing. The gait measurement system of the present example embodiment has the same configuration as that of the gait measurement system of the first or second example embodiment, except that the acquisition device and the transmission device are excluded.

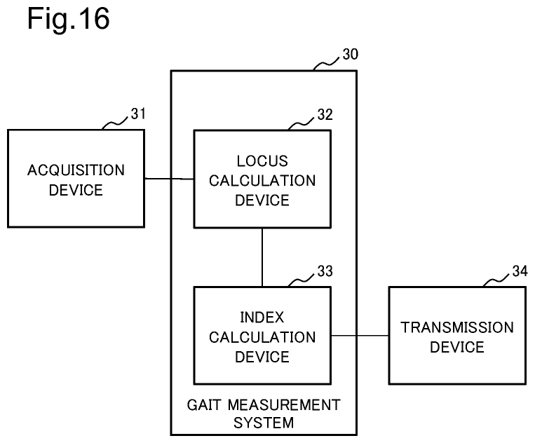

FIG. 16 is a block diagram showing the configuration of a gait measurement system 30 of the present example embodiment. As shown in FIG. 16, the gait measurement system 30 includes a locus calculation device 32 and an index calculation device 33. The gait measurement system 30 is connected to an acquisition device 31 and a transmission device 34. The acquisition device 31 and the transmission device 34 have the same configuration as the acquisition device 11 and the transmission device 14 of the gait measurement system 1 of the first example embodiment. For example, the acquisition device 11 includes an inertial measurement unit.

The locus calculation device 32 is connected to the acquisition device 31. The locus calculation device 32 is also connected to the index calculation device 33. The locus calculation device 32 receives acceleration data from the acquisition device 31. The locus calculation device 32 detects at least one gait phase from the acceleration data measured by the inertial measurement unit, and calculates velocity data by subjecting the acceleration data to time integration. The locus calculation device 32 calculates correction amounts associated with each of the gait phases by using the gait phases and the velocity data, and calculates correction velocity data by subtracting the correction amounts from the velocity data associated with each of the gait phases. The locus calculation device 32 calculates locus data by subjecting the calculated correction velocity data to time integration. The locus calculation device 32 transmits the calculated locus data to the index calculation device 33.

The index calculation device 33 is connected to the locus calculation device 32. The index calculation device 33 is also connected to the transmission device 34. The index calculation device 33 receives the locus data from the locus calculation device 32. The index calculation device 33 calculates gait indexes, using the received locus data. For example, the index calculation device 33 calculates a stride length, a gait velocity, and the like as the gait indexes. The index calculation device 33 transmits the calculated gait indexes to the transmission device 34. Alternatively, the gait indexes calculated by the index calculation device 33 may be transmitted to the outside.

An example configuration of the gait measurement system 30 has been described so far. However, the configuration of the gait measurement system 30 shown in FIG. 16 is merely an example, and does not limit the configuration of the gait measurement system 30 of the present example embodiment to the same configuration as above.

As described above, a gait measurement system of the present example embodiment includes a locus calculation device and an index calculation device. The locus calculation device detects at least one gait phase from acceleration data measured by an inertial measurement unit, and calculates velocity data by subjecting the acceleration data to time integration. The locus calculation device calculates correction amounts associated with each of the gait phases by using the gait phases and the velocity data, and calculates correction velocity data by subtracting the correction amounts from the velocity data associated with each of the gait phases. The locus calculation device calculates locus data by subjecting the calculated correction velocity data to time integration. The index calculation device calculates gait indexes, using the locus data calculated by the locus calculation device.

With the gait measurement system of the present example embodiment, it is possible to eliminate drift that shows different characteristics between a stance phase and a swing phase, and thus, gait can be measured with high accuracy.

(Hardware)

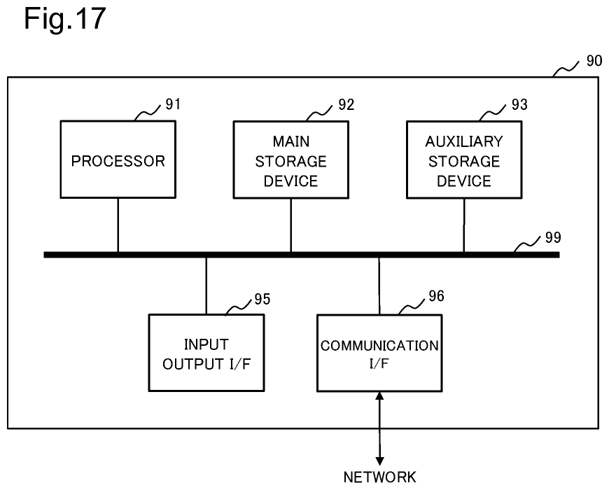

A hardware configuration for performing processes in a locus calculation device and an index calculation device according to each example embodiment of the present invention is now described, with an information processing apparatus 90 in FIG. 17 being taken an example. However, the information processing apparatus 90 shown in FIG. 17 is merely an example configuration for performing processes in the locus calculation device and the index calculation device of each example embodiment, and does not limit the scope of the present invention.

As shown in FIG. 17, the information processing apparatus 90 includes a processor 91, a main storage device 92, an auxiliary storage device 93, an input/output interface 95, and a communication interface 96. In FIG. 17, “interface” is abbreviated as “I/F”. The processor 91, the main storage device 92, the auxiliary storage device 93, the input/output interface 95, and the communication interface 96 are connected to one another via a bus 99 in such a way as to be capable of data communication. The processor 91, the main storage device 92, the auxiliary storage device 93, and the input/output interface 95 are also connected to a network such as the Internet or an intranet via the communication interface 96.

The processor 91 loads a program stored in the auxiliary storage device 93 or the like into the main storage device 92, and executes the loaded program. In the present example embodiment, a software program installed in the information processing apparatus 90 may be used. The processor 91 performs the processes with the locus calculation device and the index calculation device according to the present example embodiment.

The main storage device 92 has a region in which a program is loaded. The main storage device 92 may be a volatile memory such as a dynamic random access memory (DRAM), for example. Also, a nonvolatile memory such as a magnetoresistive random access memory (MRAM) may be formed and added as the main storage device 92.

The auxiliary storage device 93 stores various kinds of data. The auxiliary storage device 93 is formed with a local disk such as a hard disk or a flash memory. However, the main storage device 92 may be designed to store various kinds of data, and the auxiliary storage device 93 may not be provided.

The input/output interface 95 is an interface for connecting the information processing apparatus 90 to peripheral devices. The communication interface 96 is an interface for connecting to an external system or device via a network such as the Internet or an intranet, in accordance with standards or specifications. The input/output interface 95 and the communication interface 96 may be shared as an interface for connecting to an external device.

An input device such as a keyboard, a mouse, or a touch panel may be connected to the information processing apparatus 90 as needed. These input devices are used for inputting information and settings. In a case where a touch panel is used as an input device, the display screen of a display device may also serve as the interface with the input device. Data communication between the processor 91 and the input device may be mediated by the input/output interface 95.

The information processing apparatus 90 may also be equipped with a display device for displaying information. In a case where a display device is provided, the information processing apparatus 90 preferably includes a display controller (not shown) for controlling display on the display device. The display device may be connected to the information processing apparatus 90 via the input/output interface 95.

The information processing apparatus 90 may also be equipped with a disk drive, if necessary. The disk drive is connected to the bus 99. Between the processor 91 and a recording medium (a program storage medium) that is not shown in the drawing, the disk drive mediates reading data or a program from the recording medium, writing of results of processing performed by the information processing apparatus 90 into the recording medium, and the like. The recording medium can be formed with an optical recording medium such as a compact disc (CD) or a digital versatile disc (DVD), for example. Alternatively, the recording medium may be formed with a semiconductor recording medium such as a universal serial bus (USB) memory or a secure digital (SD) card, a magnetic recording medium such as a flexible disk, or some other recording medium.

An example hardware configuration for achieving the locus calculation device and the index calculation device according to each example embodiment of the present invention has been described so far. However, the hardware configuration shown in FIG. 17 is merely an example hardware configuration for performing arithmetic processes in the locus calculation device and the index calculation device according to each example embodiment, and does not limit the scope of the present invention. A program for causing a computer to perform processes related to the locus calculation device and the index calculation device according to each example embodiment is also included in the scope of the present invention. Further, a program storage medium storing a program according to each example embodiment is also included in the scope of the present invention.

The components of the locus calculation device and the index calculation device of each example embodiment may be combined as appropriate. The components of the locus calculation device and the index calculation device of each example embodiment may be formed by software, or may be formed by circuitry.

While the present invention has been particularly shown and described with reference to example embodiments thereof, the present invention is not limited to these example embodiments. It will be understood by those of ordinary skill in the art that various changes in form and details may be made therein without departing from the spirit and scope of the present invention as defined by the claims.

### REFERENCE SIGNS LIST

1, 2 gait measurement system
11, 21, 31 acquisition device
12, 22, 32 locus calculation device
13, 23, 33 index calculation device
14, 24, 34 transmission device
121, 221 gait phase detection unit
122, 222 first integration unit
123, 223 correction amount calculation unit
124, 224 subtraction unit
125, 225 second integration unit
131, 231 first correction amount calculation unit
132, 232 second correction amount calculation unit
220 coordinate transform unit

What is claimed is:

1. A gait measurement system comprising:
at least one memory storing instructions; and
at least one processor connected to the at least one memory and configured to execute the instructions to:
detect at least one gait phase from acceleration data measured by an inertial measurement unit,
calculate velocity data by subjecting the acceleration data to time integration,
calculate correction amounts associated with each of the gait phases using the gait phases and the velocity data,
calculate correction velocity data by subtracting the correction amounts from the velocity data associated with each of the gait phases,
calculate locus data by subjecting the calculated correction velocity data to time integration; and
calculate a gait index, using the locus data calculated by the locus calculation device.

2. The gait measurement system according to claim 1, wherein
the at least one processor is configured to:
acquire the acceleration data from the inertial measurement unit, and detect the at least one gait phase, using the acquired acceleration data;
acquire the acceleration data from the inertial measurement unit, and calculate the velocity data by subjecting the acquired acceleration data to time integration;
calculate the correction amounts associated with each of the gait phases, using the gait phases and the velocity data;
calculate the correction velocity data by subtracting the correction amounts associated with each of the gait phases from the velocity data; and
calculate the locus data by subjecting the correction velocity data to time integration.

3. The gait measurement system according to claim 2, wherein
the at least one processor is configured to
detect a stance phase and a swing phase as the gait phases, and
calculate a first correction amount for making velocity bias in the stance phase zero and a second correction amount for reducing drift in the swing phase, and
calculate the correction velocity data by subtracting the calculated first correction amount and second correction amount from the velocity data.

4. The gait measurement system according to claim 3, wherein
the at least one processor is configured to detect
a period from heel contact to toe detachment as the stance phase, and
a period from toe detachment to heel contact as the swing phase.

5. The gait measurement system according to claim 1, wherein
the at least one processor is configured to:
acquire the acceleration data and angular velocity data from the inertial measurement unit,
perform coordinate transform to transform the acceleration data into correction acceleration data in a world coordinate system, using the acquired angular velocity data;
detect the at least one gait phase, using the correction acceleration data;
calculating the velocity data by subjecting the correction acceleration data to time integration;
calculate the correction amounts associated with each of the gait phases, using the gait phases and the velocity data;
calculate the correction velocity data by subtracting the correction amounts associated with each of the gait phases from the velocity data; and
calculate the locus data by subjecting the correction velocity data to time integration.

6. The gait measurement system according to claim 1, wherein
the at least one processor is configured to:
acquire the acceleration data and angular velocity data from the inertial measurement unit,
perform coordinate transform to transform the acceleration data into correction acceleration data in a world coordinate system, using the acquired angular velocity data;
acquire the acceleration data from the inertial measurement unit,
detect the at least one gait phase, using the acquired correction acceleration data;
calculate the velocity data by subjecting the correction acceleration data to time integration;
calculate the correction amounts associated with each of the gait phases, using the gait phases and the velocity data;
calculate the correction velocity data by subtracting the correction amounts associated with each of the gait phases from the velocity data; and
calculate the locus data by subjecting the correction velocity data to time integration.

7. The gait measurement system according to claim 5, wherein
the at least one processor is configured to perform coordinate transform to transform the acceleration data into correction acceleration data in a world coordinate system, using a technique of Madgwick.

8. The gait measurement system according to claim 1, further comprising
an acquisition device configured to include the inertial measurement unit, and acquire at least the acceleration data with the inertial measurement unit; and
a display device configured to display the gait index calculated by the index calculation device.

9. A gait measurement method comprising:
detecting at least one gait phase from acceleration data measured by an inertial measurement unit;
calculating velocity data by subjecting the acceleration data to time integration;
calculating correction amounts associated with each of the gait phases, using the gait phases and the velocity data;
calculating correction velocity data by subtracting the correction amounts from the velocity data associated with each of the gait phases;
calculating locus data by subjecting the calculated correction velocity data to time integration; and
calculating a gait index, using the calculated locus data.

10. A program storage medium storing a program for causing a computer to perform:
a process of detecting at least one gait phase from acceleration data measured by an inertial measurement unit;
a process of calculating velocity data by subjecting the acceleration data to time integration;
a process of calculating correction amounts associated with each of the gait phases, using the gait phases and the velocity data;
a process of calculating correction velocity data by subtracting the correction amounts from the velocity data associated with each of the gait phases;
a process of calculating locus data by subjecting the calculated correction velocity data to time integration; and
a process of calculating a gait index, using the calculated locus data.
